(19) United States
(12) Patent Application Publication
(10) Pub. No.: US 2023/0404385 A1
(43) Pub. Date: Dec. 21, 2023

(54) VISUAL FIELD TESTING

(71) Applicant: HEADS STOCKHOLM AB, Stockholm (SE)

(72) Inventor: Hans-Peter Kurz, Stockholm (SE)

(21) Appl. No.: 18/265,095

(22) PCT Filed: Dec. 2, 2021
(86) PCT No.: PCT/SE2021/051195

§ 371 Date: Jun. 2, 2023

(30) Foreign Application Priority Data
Dec. 3, 2020 (SE) 2051412-1

(51) Int. Cl. A61B 3/024; A61B 3/113; A61B 3/00; A61B 3/11

(52) U.S. Cl. CPC A61B 3/024; A61B 3/113; A61B 3/0041; A61B 3/112

(57) ABSTRACT

The present disclosure relates to a device for visual field testing 11, which maps eye function of a test subject, the device comprising a display unit 17 displaying stimuli 23 to the test subject, and an eye tracking unit 19 detecting eye motions in response to the displayed stimuli. The display unit 17 displays at least a first 23 and a second 25 peripheral stimulus at different sectors in relation to a point of fixation 27 at the same time and the eye tracking unit determines which of the first and second stimulus is first gazed upon by the test subject. This allows different parts of the retina to compete with regards to retinal sensitivity and allows for a precise and efficient visual field testing.

### FIELD OF THE INVENTION

The present disclosure relates to a device for visual field testing, mapping eye function of a test subject, the device comprising a display unit for displaying stimuli to the test subject, an eye tracking unit for detecting eye motions in response to the displayed stimuli, and an analyzing unit for determining a visual field characteristics based on the displayed stimuli and a corresponding eye tracking response.

### BACKGROUND OF THE INVENTION

One example of such a device is described in WO-2919/169322-A1, where visual field testing is carried out with the assistance of eye tracking to provide test subject responses to stimuli. Usually, tests are carried out by testing at which contrast or brightness the test subject sees a stimulus that is presented in a position peripheral to a point of fixation. Other variations exist as will be discussed.

One problem associated with devices and methods for determining visual field characteristics is that the examination usually takes a considerable time, as a large number of points within the visual field need be tested. This is tiring for the test subject meaning that results may deteriorate during the examination process, and that the general status, such as stress, sleep deprivation etc. of the test subject, can affect the result to a great extent as the subject may react slower to stimulus, for instance. This means that the test can be too unreliable, for instance when tests are conducted repeatedly to assess age-related visual conditions.

### SUMMARY OF THE INVENTION

One object of the present disclosure is therefore to provide a device that tests visual field in a more reliable way. This object is achieved by means of a device as defined in claim 1. More specifically, in a device of the initially mentioned kind, the display unit is configured to simultaneously display at least a first and a second stimulus at different sectors in relation to a point of fixation, and the eye tracking unit or the analyzing unit is configured to determine which of the first and second stimulus is first gazed upon by the test subject. This means that each point is tested in relation to another point in the visual field, or optionally in relation to a point in the visual field of the test subject's other eye. In this way a very detailed and more reliable visual field mapping can be obtained.

In addition to the first and second peripheral stimuli, the display unit may be configured to display a central stimulus at the point of fixation. Alternatively, a permanent central stimulus may be formed on the display.

Typically, the display unit may be configured to display the first and second peripheral stimuli at locations separated by a minimum angle φ in the range between 15 and 180 degrees as seen from the point of fixation or central stimulus. This facilitates the determination of which peripheral stimulus was detected.

The peripheral stimuli may be presented simultaneously, or at least partly overlapping in time. In any case they should be presented within a common time frame shorter than 250 ms to enable the competing between the first and second stimuli.

The first and second peripheral stimuli may be presented to the same eye to obtain the relative sensitivity strength between two areas in the same retina, or the first peripheral stimulus may be presented to a first eye and the second peripheral stimulus to the second eye to establish also a relative sensitivity between the eyes.

The first and second peripheral stimuli have mutually different optical properties at a given point of time. Typically, this makes either stimulus easier to detect and allows different areas of the retina, with different sensitivities, to be tested against each other. The different optical properties may include one or more in the group comprising: size, brightness, contrast, shape color, and variability.

Alternatively, or in combination therewith, at least one of the first and second peripheral stimuli may move from a location corresponding to region with projected lower retinal sensitivity to a location corresponding to a region with higher retinal sensitivity. Then, the eye reacts to the stimulus that first reaches an area with sufficiently high sensitivity. The peripheral stimuli may typically move from the exterior of the visual field towards the center thereof. It is however also possible to let one of the first and second peripheral stimuli move out of a region with reduced sensitivity, such as a blind spot or scotoma.

Typically, the display is configured to increase the visibility of the first and second stimuli over time to trigger a response from the tested subject.

In some cases, three or more peripheral stimuli may be provided within the common time frame.

The eye tracking device is configured to detect the pupil size of at least one eye and to adjust optical properties of at least one stimulus based on said pupil size. As an alternative, the pupil size may be recorded together with the other measured data.

It is also possible to let the eye tracking device detect the pupil size of one eye and to make the display device control the amount of light to which the other eye is exposed in such a way that said pupil size of the first eye reaches a desired diameter. This can be used to make sure that testing is carried out at a constant pupil size also over several testing sessions.

It is also possible to let the eye tracking device detect the pupil size of at least one eye and make the display device control the background light shown to the at least one eye in such a way that said pupil size of one or both eyes reach a desired diameter. The stimuli may then be adjusted accordingly.

To generally decrease the visibility of stimuli, it is possible to provide, with the display device, the background, upon which the peripheral stimulus is presented, as a fluctuating pattern. This may be done in a random or pseudo-random fashion.

The display unit and the eye tracking unit may be included in a wearable device which is attached to the head of the test subject.

The present disclosure further considers a non-transitory computer-readable medium for mapping eye function of a test subject over a visual field, the medium comprising instructions stored thereon that when executed on a processor cause the processor to: display stimuli to the test subject using a display unit, detecting eye motions in response to the displayed stimuli by means of an eye tracking unit, and analyzing a visual field characteristics based on the displayed stimuli and a corresponding eye tracking response. The instructions cause the display unit to display at least a first and a second peripheral stimuli at different sectors in relation to a point of fixation, and in a common time frame, and the to determine, using an eye tracking unit, which of the first and second stimulus is first gazed upon by the test subject.

Such software instructions when stored and executed as indicated provide the same advantages as the above device and may be varied accordingly.

### BRIEF DESCRIPTION OF THE DRAWINGS

FIG. 1A illustrates a top view of a hill of vision, HoV, with indicated isopters.

FIG. 1B illustrates a perspective view of the HoV of FIG. 1A.

FIG. 1C illustrates a cross section through the HoV of FIGS. 1A, 1B.

FIG. 2A illustrates a basic hardware setup where the present disclosure can be realized.

FIG. 2B and 2C illustrate arrangements where both eyes of a tested subject are tested at the same time.

FIG. 3 illustrates a basic method according to the present disclosure.

FIG. 4 illustrates an example with stationary stimuli.

FIG. 5 illustrates an alternative example with moving stimuli.

FIG. 6 illustrates one option for outputting data and a testing sequence.

### DETAILED DESCRIPTION OF THE EXEMPLARY EMBODIMENTS

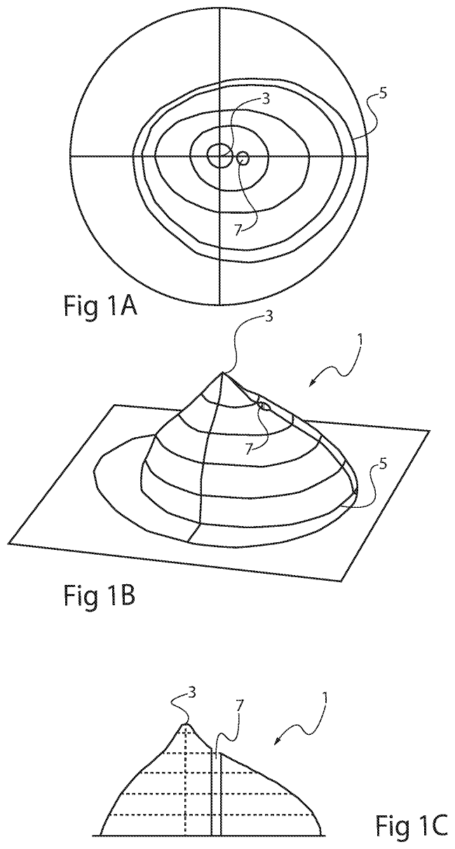

The present disclosure relates in general to devices for mapping the eye function of a test subject, specifically visual field testing. By visual field is meant the space, eccentric to the point of fixation, where the human eye can detect a stimulus. Within the visual field, the ability to detect stimuli varies, and this may be mapped as a Hill of Vision 1, HoV, as illustrated in FIGS. 1A-1C.

The Hill of Vision 1 corresponds directly to the sensitivity of a human retina over its surface. As illustrated, the sensitivity of a healthy retina is higher at the center 3 thereof and decreases towards the periphery until reaching the border 5 of the field of vision, FoV. Even a healthy retina includes a blind spot 7 within the retina, typically where the optic nerve reaches the retina.

Vision impairments associated with different retinal disorders changes the HoV, typically over a long period of time.

One known disorder is Glaucoma. Glaucoma is an eye disease with damage to the optic nerve and retina. This causes vision loss and a decreased HoV.

Another such disorder is Age-Related Macula Degeneration, AMD. AMD is fairly common among older people and causes the otherwise very sensitive macula at the center of the field of vision to become damaged, leading to blurred areas or even blank spots in the subject's central vision. This may progress more or less rapidly until everyday tasks such as driving, reading or using computers become difficult.

Detailed testing of the retinal sensitivity is important to assess the progression of retinal disorders and to determine suitable treatments and to select useful vision aids to improve eyesight, e.g. projecting incoming light on healthy areas of the retina.

Typically, visual field testing has been carried out by letting the test subject first fixate his gaze on a central stimulus point which is displayed to the test subject. Then, a peripheral stimulus is produced in a peripheral position with regard to the central stimulus point. The test subject provides feedback when being able to see the peripheral stimulus, e.g. by pressing a button or orally telling a test administrator about being able to see the stimulus and optionally where. It has also been suggested to use eye tracking functionality to determine when the test subject carries out a saccade from the central stimulus or point of fixation to the peripheral stimulus. It has also been suggested to use eye tracking to verify that the test subject really fixates the central stimulus prior to producing the peripheral stimulus, e.g. in order to make sure that the test subject does not cheat.

Different sectors of the visual field may be sequentially tested, and the peripheral stimulus may be produced in different ways. In a static test, a peripheral stimulus is provided at a fixed point in relation to the central stimulus. This peripheral stimulus may then become more visible, until the test subject responds thereto. For instance, the peripheral stimulus may have parameters such as size, brightness or contrast, or a combination thereof, increased until being seen by the test subject. The parameters at the time the test subject sees the peripheral stimulus indicates the retina's corresponding sensitivity at that point.

Another option is a so-called kinetic test, where a stimulus is not fixed, but moves from the periphery of the visual field and towards the center thereof. The point where the tested subject sees the stimulus indicates the tested subject's retinal sensitivity in the sector, in relation to the central stimulus, where the peripheral stimulus appears. This allows for a quicker, but coarser, test as sectors rather than individual points are tested. It is however possible to combine those two options e.g. by repeating a kinetic test with a different peripheral stimulus brightness, for instance. The result may then be an isopter plot as shown in FIG. 1A, where lines follow a path around the visual field where the retinal sensitivity is the same. A static test on the other hand may be used to map an HoV with arbitrary resolution, although a large number of testing points may require a very long testing procedure.

The relatively long testing process needed to establish a mapping of the retinal sensitivity of a test subject implies that in many cases the test becomes unreliable. After a while the test subject becomes tired and may be easily distracted, which means, for instance, that he after a while may react more slowly to a stimulus at a location and a visibility (size, brightness, contrast) which he would have reacted quickly to in the beginning in the testing procedure. The result is an incorrectly indicated retinal sensitivity.

The present disclosure considers an improved device for visual field testing

### Basic Setup

The present disclosure uses an eye tracking functionality that measures the movements of a user's eye or eyes. Usually one eye at a time is tested although in some cases, as will be described, it may be desired to test both eyes simultaneously or alternatingly.

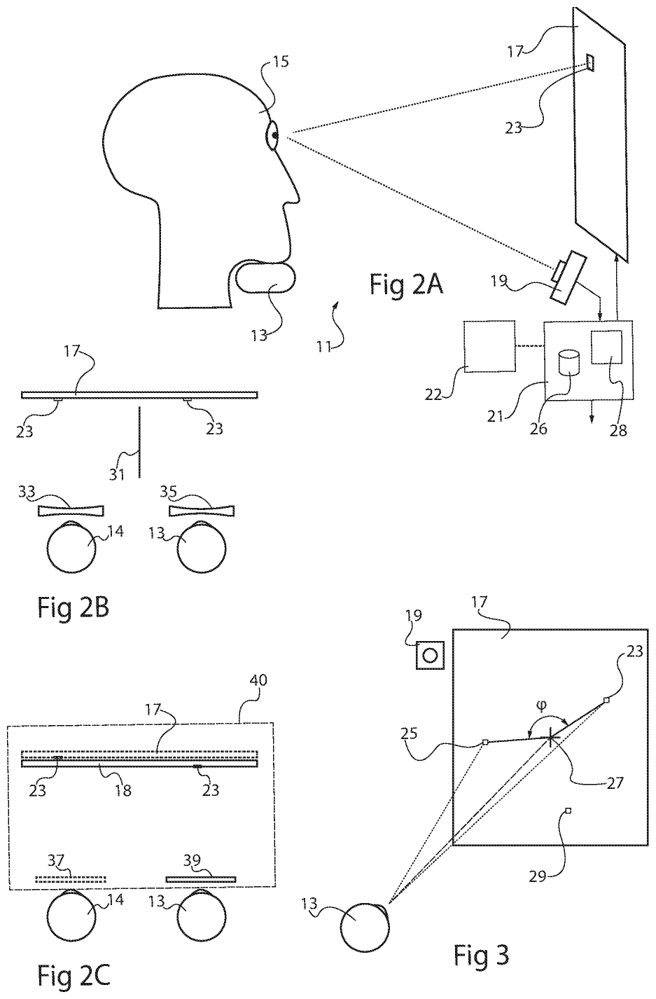

FIG. 2A schematically shows a basic arrangement 11 for carrying out tests. The arrangement may optionally include a head rest 13 where the tested subject's head 15 can rest during testing. The test person watches a display 17 on which various visual stimuli may be produced. An eyetracker 19 is used to track the user's eye movements, and an analyzing unit 21 compares the provided stimulus with the eyetracking response to determine a corresponding eye metric and/or other information. The analyzing unit may communicate with a database 22 which contains e.g. corresponding metrics from persons e.g. with known deficiencies or without deficiencies etc. in order to provide a more elaborated result. The analyzing unit typically includes a computer-readable medium 26 comprising instructions stored thereon that when executed on a processor 28 cause the processor to carry out the actions of the device 11 as a whole.

It should be noted that the arrangement in FIG. 2A is very simplified. A real implementation may include multiple screens, curved screens, wavelength selective mirrors, head motion sensors etc.

The eyetracking may be based on any eyetracking technology, such as so-called bright and/or dark pupil measurements, iris detection, sclera movement observations or glint measurements or a combination thereof, as per se is well known in the art.

It should be noted that the components of FIG. 2A could be integrated in a virtual reality, VR, headset 40 (cf. FIG. 2C), for instance, which is strapped on the tested subject's head. FIG. 2B illustrates one example where different content is displayed to each eye 13, 14. The display device 17 is segmented into two portions by means of a wall 31 in between the eyes 13, 14, such that stimulus 23 displayed to one eye is not visible to the other. Lenses 33, 35 can be provided at the eyes to compensate for any cylindrical or spherical refractive errors of the eyes or to allow the display device 17 to be located at a short distance.

Another option is disclosed in FIG. 2C where two display devices 17, 18 are stacked and provide different content over a common surface, and where different filters 37, 39 are to each eye 13, 14 such that they each can view either of the display devices 18, 17. This can be achieved with color- or polarization filters, for instance.

In the present disclosure there is provided, again with reference to FIG. 2A, a device for visual field testing 11, that maps the eye function of a test subject 15. Typically, a hill of vision is outputted as a result. The results may also be visualized in other diagram formats, for instance in a Bebie curve format. The results may also be directly visualized in structural recordings as a fundus picture or an OCT scan of the tested eye. The results may also be added into standard kinetic or static visualization plots such as derived for instance from the Goldmann perimeter or the Humphrey field analyzer.

The device comprises a display unit 17 for displaying stimuli 23 to the test subject 15, whose response thereto is determined at least partly using an eye tracking unit 19, detecting eye motions in response to the displayed stimuli. An analyzing unit 21 determines a visual field characteristic based on the displayed stimuli and a corresponding eye tracking response.

With reference to FIG. 3, the test subject is asked to view a point of fixation, which in the illustrated case is a central stimulus 27 displayed by the display unit 17. It would be possible to provide a visible point of fixation in other ways, for instance with a mark on top of the display unit screen. It is possible to use the eye tracking device 19 to verify that the tested subject continues to view the point of fixation until another stimulus is provided to avoid for instance that the tested subject searches the screen for a stimulus, thereby giving a corrupted result in the following sequence steps. If displayed by the display unit, the central stimulus need not be static, it may be moved and/or dynamically alter its appearance to more readily maintain the attention of the tested subject.

Then, the display unit 17 displays stimuli 23, 25, 29 at a distance from the point of fixation 27. If the test subject's retina is sensitive enough to detect a stimulus, he will make a saccade (gaze movement) towards the detected stimulus which can be detected by the eye tracking device 19.

In the present disclosure, at least a first 23 and a second 25 peripheral stimulus at different sectors in relation to a point of fixation 27. Those are provided more or less simultaneously or at overlapping time frames, or at least within a common time frame. This means that there exists a competition between the first and second stimuli 23, 25, and this competition is much less influenced by the test subject becoming tired, for instance. Thereby, the relative sensitivity at two different locations of the HoV can be very reliably determined. This is done by the eye tracking unit 19, or the analyzing unit 21, determining which of the first and second stimulus is first gazed upon by the test subject. This can be done with great certainty, especially if a reasonable angular separation is provided between the first and second stimuli 23, 25. Preferably, the first and second peripheral stimuli 23, 25 are displayed at locations separated by a minimum angle φ in the range between 15 and 180 degrees, although a useful result could be accomplished with a smaller separation.

As mentioned, the first and second peripheral stimuli 23, 25 may be presented simultaneously or partly overlapping in time. It may be enough, though that the first and second peripheral stimuli 23, 25 are presented within a common time frame shorter that 250 ms, meaning that the end of the displaying of one stimulus and the beginning of the displaying of another are not separated more than 250 ms.

It is possible to provide more than two peripheral stimuli, such as the additional third stimulus 29 shown in FIG. 3, within this time frame. This allows for testing schemes testing several locations on the retina simultaneously.

As mention, it is possible to test the eyes of a test subject one at a time. This can be done by occluding one eye as is traditionally made, but the same effect can be achieved by simply display the stimuli at a screen or screen portion associated with one of the eyes. It may be that a complete test is carried out at one eye at a time, but it is also possible to repeatedly switch between testing the right and left eyes, allowing one eye to rest a while. The multiple stimuli 23, 25 as shown in FIG. 3 can thus be presented to the same eye 13.

The present disclosure however also proposes presenting the peripheral stimuli to both eyes within the same time frame. Then, for instance, the first peripheral stimulus 23 is presented to the right eye 13 while at the same time the second peripheral stimulus 25 is presented to second eye 14. By detecting which eye that first detects its stimulus, it is possible also to rank retina areas of the left and right eye against each other. This provides a further set of useful information as the relative HoV of the right and left eyes can be determined to some extent.

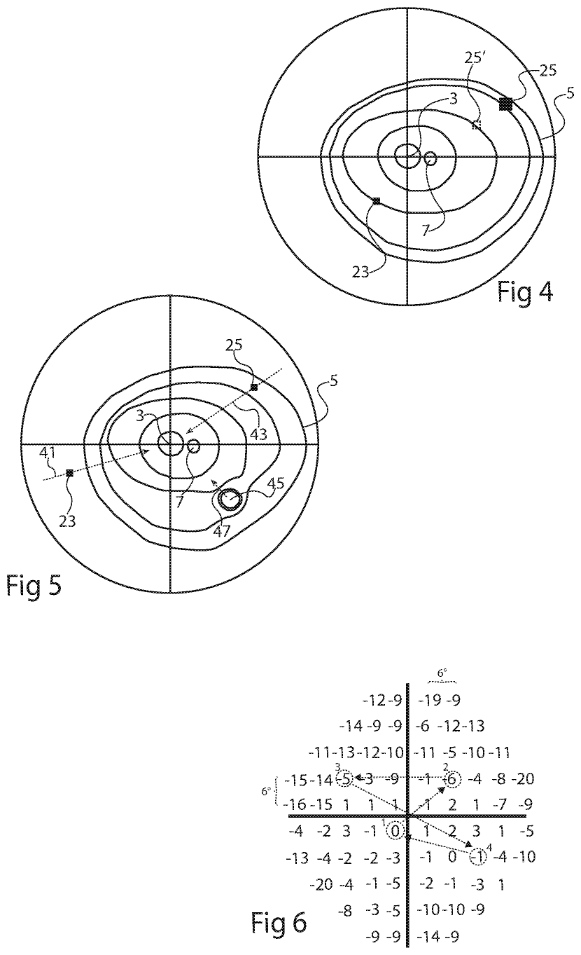

FIG. 4 illustrates an example of a static approach for visual field testing according to the present disclosure. In this case, a first stimulus 23 is displayed in the third quadrant and a second stimulus 25 is displayed in the first quadrant. The test in these locations can be repeated a number of times. Typically, the stimuli may grow or have an increasing contrast over time until the eye reacts on one of them. The first and second peripheral stimuli 23, 25 further may have mutually different optical properties at a given point of time, as illustrated for instance with different sizes. This means that if the eye makes a saccade from the center 3 coinciding with the point of fixation to the smaller first stimulus 23, the corresponding location of the retina is most certainly more sensitive than the corresponding location of larger stimulus 25 as is also indicated by the isopters of those locations.

The stimuli may also be identical in terms of visibility. It is therefore possible to test with stimuli at different location which increase in visibility until one is detected at which time the corresponding location on the retina is rated as stronger than the other. By repeating such a procedure at different locations a number of times the relative HoV can be resolved.

As indicated with the alternative location and size of the second stimulus 25′, it could thus also be presented with the same size as the first stimulus 23. If at consecutive tests at this location the tested subject alternatingly chooses both stimuli, it can be assumed that the sensitivity is about the same at the corresponding retinal locations, i.e. that those locations are situated at the same isopters, as indicated in FIG. 4.

It would be possible just to display the stimuli at the two locations and, detect whether or not the tested subject detects either stimulus. Typically, however, in the static example, where the stimuli do not move, the visibility may increase gradually until the tested subject detects either of the stimuli. Further, as mentioned, the visibility of the stimuli may differ between the location at any given instant. The visibility may increase in different ways during a testing. For instance the brightness and or contrast of a stimulus may increase over a period of time, and subsequently the stimulus may begin to flash, typically with a frequency higher than 1 Hz, in order to determine whether the eye can detect the stimulus.

One option for increasing the visibility of a stimulus is of course to increase its size. Another option is to increase the brightness and/or contrast with which the stimulus is displayed.

It would also be possible to increase the visibility by changing the shape of the stimulus into one the blends into the background to a lesser extent. Further, variability of the stimulus can be changed, for instance that it fluctuates or flashes or that a pattern moves over the surface of the stimulus.

One such way of introducing a variability is to introduce a so-called Frequency Doubling Technology, FDT, pattern with a sinusoidal grating that provides a flicker, for instance at 18 Hz, such as used in Humphrey Matrix FDT perimetry.

The visibility of the two or more stimuli can also be changed by altering the background, e.g. to increase the contrast of the stimuli. Also, the background may have a variability that is altered.

Note that the above-mentioned methods for increasing the visibility can be combined in different ways.

Another option is to use a kinetic testing approach. Then, a moving stimulus is used, which travels over the display, typically from an area believed to be associated with lower or no retinal sensitivity and towards an area with a higher retinal sensitivity. This makes a relatively quick testing possible. Rather than testing a point in the visual field, a path in the field is tested, for instance in a sector with regard to the central point 3 which may coincide with the point of fixation. Thus, a sector may for instance be tested by allowing a stimulus travel repeatedly from the periphery of the visual field, and towards the center thereof. By increasing the size of the stimulus in between repetitions and detecting the eye's response, a measure of the HoV slope at this sector can be obtained.

The above described kinetic approach can be improved according to the present disclosure with two or more simultaneously displayed stimuli as described earlier. Thus, as illustrated in FIG. 5, at least one of the first 23 and second 25 peripheral stimuli move along paths 41, 43 from a location corresponding to region with projected lower retinal sensitivity to a location corresponding to a region with higher retinal sensitivity. These paths may as shown typically stretch from the exterior of the visual field towards the center 3 thereof which may coincide with the point of fixation. Depending on which stimulus the eye or eyes responds to, it can be determined which of the stimuli has reached the highest area in terms of retinal sensitivity. In the illustrated case, the paths 41, 43 are straight and directed towards the visual field center, although neither of these features are by any means necessary. The paths may well be curved, and the center of the visual field may not even be known when testing begins.

It would also be possible to make one of the first 23 and second 25 peripheral stimuli move along a path 47 from another location 45 with low retinal sensitivity such as a scotoma or the aforementioned blind spot 7. By a scotoma is meant a degrading portion of the retina having very low or no sensitivity, a feature that is frequent in some retina degrading conditions.

The moving stimuli may have a constant appearance, or may have a varying, typically increasing visibility over time to more quickly trigger a response from the tested subject. The visibility may be varied in the same ways as described above with the stationary stimuli. One possible strategy to quantify the size of a scotoma is to grow the size of the stimuli within an area where a scotoma is expected.

The device may thus, as illustrated with an example in FIG. 6, test different points on a retina against each other in a sequence as illustrated where a position close to the center in the third quadrant is first is tested against a point in the first quadrant, which is then tested against a point in the second quadrant and so forth. This establishes relative sensitivities for the points tested. Eventually the device may return to the first point, for instance, to verify the intervening results. It is thus not necessary to test all points against each other which would be very time consuming. Instead a rating may be established, similar to an ELO-rating, where eventually a rating value is established for each point or a subset of points depending on a number of point to point measurements.

It is possible at the starting point or any intervening point to carry out a measurement according to known art, for instance, to establish an absolute value of the local sensitivity that can be used to resolve the HoV as a whole.

The shown data indicates severe macular degeneration, as seen especially in the first and second quadrants. The rows and columns in the matrix of points are typically separated by 6 degrees, as indicated in FIG. 6.

The outcome of a visual field measurement can be influenced the pupil size, which determines the amount of light entering the eye and the sharpness of the image projected on the retina. Therefore, it may be useful to determine the size of the pupil. This can be done by means of the eye tracking device that is for eye tracking purposes already records images of the eye.

The pupil size may be used to adjust determined visual field measurements e.g. using a lookup table. It is also possible to adjust optical properties of a stimulus based on the pupil size.

In a further example, the eye tracking device may detect the pupil size of one eye and may control that eye's pupil size by adjusting the light to which the other eye is exposed. This can be done, for instance, in a setup as illustrated in FIG. 2B where the right and left eye are exposed to different parts of the display device, the brightness of which can be controlled independently. In that way, the other eye's pupil size can be controlled until it reaches a desired diameter.

The display device may provide the background, upon which the peripheral stimulus is presented, as a fluctuating pattern. This pattern may be fluctuating in a random or pseudo-random fashion. Vice versa the stimuli may be non-flickering, but the background may contain some random flickering homogenously distributed pattern in order to reduce the visibility of the stimuli. This increases the sensitivity of the device.

The present disclosure is not restricted to the above disclosed examples and may be varied and altered in different ways within the scope of the appended claims.

1. A device for visual field testing, mapping eye function of a test subject, the device comprising a display unit for displaying stimuli to the test subject, an eye tracking unit for detecting eye motions in response to the displayed stimuli, and an analyzing unit for determining a visual field characteristics based on the displayed stimuli and a corresponding eye tracking response, characterized by the display unit being configured to display at least a first and a second peripheral stimulus at different sectors in relation to a point of fixation, and in a common time frame, and the eye tracking unit or the analyzing unit being configured to determine which of the first and second stimulus is first gazed upon by the test subject.

2. The device according to claim 1, wherein the display unit is configured to display a central stimulus at the point of fixation.

3. The device according to claim 1, wherein the display unit is configured to display the first and second peripheral stimuli at locations separated by a minimum angle φ in the range between 15 and 180 degrees as seen from the point of fixation.

4. The device according to claim 1, wherein the first and second peripheral stimuli are presented within a common time frame shorter than 250 ms.

5. The device according to claim 4, wherein the wherein the first and second peripheral stimuli are presented at least partly overlapping in time.

6. The device according to claim 1, wherein the first and second peripheral stimuli are presented to the same eye.

7. The device according to claim 1, wherein the first peripheral stimulus is presented to a first eye and the second peripheral stimulus is presented to the second eye.

8. The device according to claim 1, wherein the first and second peripheral stimuli have mutually different optical properties at a given point of time.

9. The device according to claim 8, wherein the different optical properties are one or more in the group comprising: size, brightness, contrast, shape, and variability.

10. The device according to claim 1, wherein at least one of the first and second peripheral stimuli move from a location corresponding to region with projected lower retinal sensitivity to a location corresponding to a region with higher retinal sensitivity.

11. The device according to claim 8, wherein said at least one of the first and second peripheral stimuli move from the exterior of the visual field towards the center thereof.

12. The device according to claim 1, wherein said at least one of the first and second peripheral stimuli move out of a finite region with reduced sensitivity, such as a blind spot or scotoma.

13. The device according to claim 1, wherein the display is configured to increase the visibility of the first and second stimuli over time to trigger a response from the tested subject.

14. The device according to claim 1, wherein three or more peripheral stimuli are provided within the common time frame.

15. The device according to claim 1, wherein the eye tracking device is configured to detect the pupil size of at least one eye and to adjust optical properties of at least one stimulus based on said pupil size.

16. The device according to claim 1, wherein the eye tracking device is configured to detect the pupil size of a first eye and the display device is configured to control the amount of light to which a second eye is exposed in such a way that said pupil size of the first eye reaches a desired diameter.

17. The device according to claim 1, wherein the eye tracking device is configured to detect the pupil size of at least one eye and the display device is configured to control the background light shown to the at least one eye in such a way that said pupil size of one or both eyes reach a desired diameter.

18. The device according to claim 1, wherein the display device is configured to provide background, upon which the peripheral stimulus is presented, as a fluctuating pattern.

19. The device according to claim 18, wherein the pattern is fluctuating in a random or pseudo-random fashion.

20. The device according to claim 1, wherein the display unit and the eye tracking unit are included in a wearable device which is attached to the head of the test subject.

21. A non-transitory computer-readable medium for mapping eye function of a test subject over a visual field, the medium comprising instructions stored thereon that when executed on a processor cause the processor to: displaying stimuli to the test subject using a display unit, detecting eye motions in response to the displayed stimuli by means of an eye tracking unit, and analyzing a visual field characteristics based on the displayed stimuli and a corresponding eye tracking response, characterized by the instructions causing the display unit to display at least a first and a second peripheral stimuli at different sectors in relation to a point of fixation, and in a common time frame, and to determine, using eye tracking unit, which of the first and second stimulus is first gazed upon by the test subject.
